(19) United States
(12) Patent Application Publication
(10) Pub. No.: US 2023/0310892 A1
(43) Pub. Date: Oct. 5, 2023

(54) ADMINISTRATION OF THERAPEUTIC RADIATION USING DEEP LEARNING MODELS TO GENERATE LEAF SEQUENCES

(71) Applicant: Siemens Healthineers International AG, Steinhausen (CH)

(72) Inventors: Shahab Basiri, Siuntio (FI); Esa Kuusela, Espoo (FI)

(21) Appl. No.: 17/708,272

(22) Filed: Mar. 30, 2022

(51) Int. Cl. A61N 5/10; G16H 20/40; G06N 3/08

(52) U.S. Cl. CPC A61N 5/1036; A61N 5/1047; A61N 5/1038; G16H 20/40; G06N 3/08

(57) ABSTRACT

A memory has stored therein a fluence map that corresponds to a particular patient and a deep learning model. The deep learning model is trained to deduce a leaf sequence for a multi-leaf collimator from a fluence map. The deep learning model comprises a neural network model that was trained, at least in part, via a reinforcement learning method. A control circuit accesses the memory and is configured to iteratively optimize a radiation treatment plan to administer the therapeutic radiation to the patient by, at least in part, generating a leaf sequence as a function of the deep learning model and the fluence map by employing a plurality of agents to each separately use the deep learning model to each generate a leaf sequence for only a single leaf pair of the multi-leaf collimator.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Determining reasonable instructions for the control points of utilized multi-leaf collimators comprises one of the more complex aspects of such optimization. Part of this complexity is owing to the fact that the leaves of such collimators may be subject to various restrictions and speed constraints that should preferably be taken into account. Additional complexity can result from needing an entirely new leaf sequencing algorithm when working with a new multi-leaf collimator, the fact that different treatment facilities may require differing tuning parameters, and/or the fact that a typical leaf sequencing algorithm concentrates on reproducing a currently-required fluence map without accounting for other complexities.

### BRIEF DESCRIPTION OF THE DRAWINGS

The above needs are at least partially met through provision of the administration of therapeutic radiation using deep learning models to generate leaf sequences described in the following detailed description, particularly when studied in conjunction with the drawings, wherein:

FIG. 1 comprises a block diagram as configured in accordance with various embodiments of these teachings; and

FIG. 2 comprises a flow diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments a memory has stored therein a fluence map that corresponds to a particular patient and a deep learning model. The deep learning model is trained to deduce a leaf sequence for a multi-leaf collimator from a fluence map. The deep learning model comprises a neural network model that was trained, at least in part, via a reinforcement learning method. A control circuit accesses the memory and is configured to iteratively optimize a radiation treatment plan to administer the therapeutic radiation to the patient by, at least in part, generating a leaf sequence as a function of the deep learning model and the fluence map by employing a plurality of agents to each separately use the deep learning model to each generate a leaf sequence for only a single leaf pair of the multi-leaf collimator.

By one approach, the aforementioned neural network model comprises a convolutional neural network model. By one approach, the aforementioned neural network model was trained, at least in part, via a supervised learning method. The neural network model may be trained using a training corpus that includes fluence maps for each of a plurality of corresponding field/control points.

By one approach, the aforementioned reinforcement learning method comprises a deep learning method. By one approach, the reinforcement learning method provides for rewarding an agent during training. Such a reward may be calculated, for example and at least in part, as a function of how well a created leaf sequence produces a target fluence.

By one approach, the aforementioned plurality of agents are each identical to one another. By another approach, such as when the multi-leaf collimator is comprised of a first kind of leaf and a second kind of leaf, wherein the first and second kind of leaves are different from one another, a first and second agent may be different from one another. In such a case, the first agent may generate leaf sequences for leaf pairs of the first kind of leaf and the second agent may generate leaf sequences for leaf pairs comprised of the second kind of leaf.

If desired, a radiation treatment platform that includes the aforementioned multi-leaf collimator can provide the therapeutic radiation to the patient as a function of the aforementioned radiation treatment plan.

So configured, the aforementioned agents can be trained based on existing cases rather than by tuning one or more corresponding heuristic algorithms. Although such training may need to be repeated for each new multi-leaf collimator, this process is straightforward and hence less burdensome than typical prior art approaches. These teachings will also readily accommodate training agents for different treatment sites to better match local needs and/or requirements. When supervised, these teachings will also accommodate training that is based, at least in part, on final plan quality rather than just on current fluence information. In many application settings, it is also anticipated that these teachings will provide improved leaf sequencing results as compared to typical prior art approaches

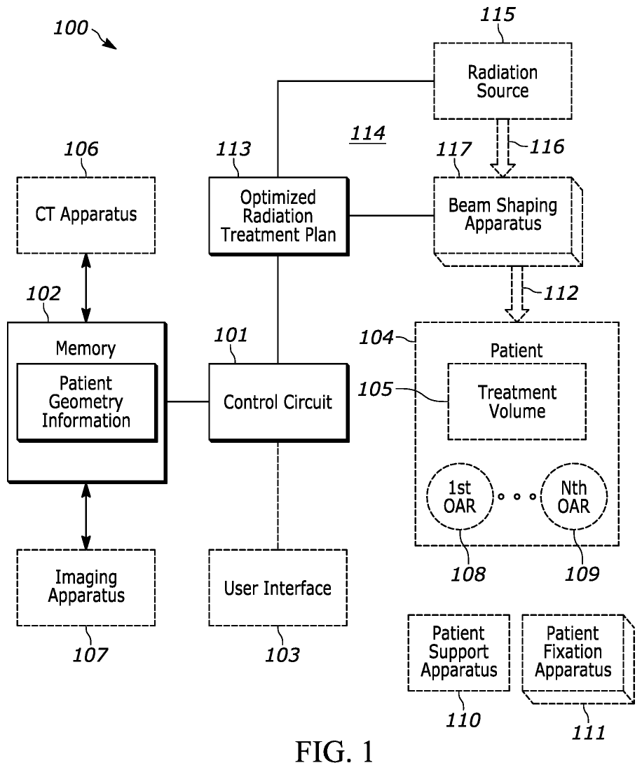

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as optimization information for a particular patient and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

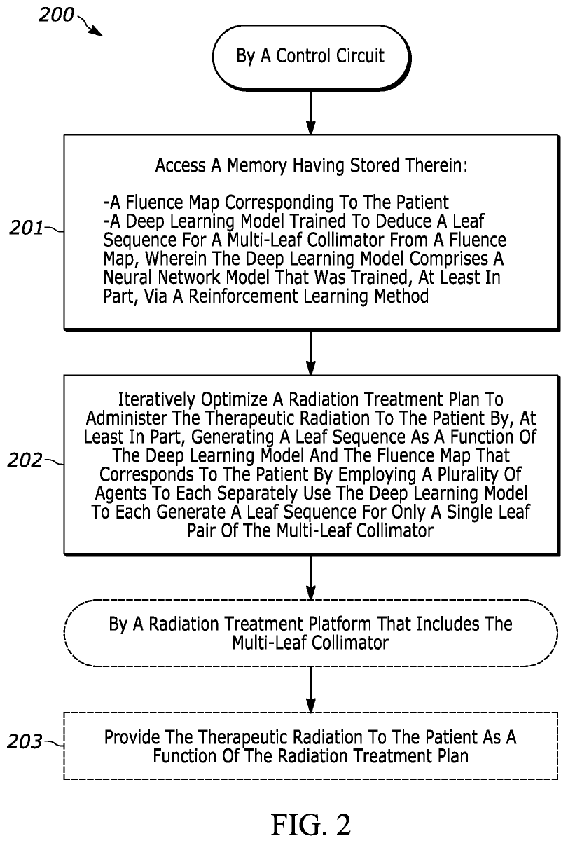

Referring now to FIG. 2, a process 200 that can be carried out, for example, in conjunction with the above-described application setting (and more particularly via the aforementioned control circuit 101) will be described. Generally speaking, this process 200 serves to facilitate generating an optimized radiation treatment plan 113 to thereby facilitate treating a particular patient with therapeutic radiation using a particular radiation treatment platform per that optimized radiation treatment plan.

At block 201, this process 200 provides for accessing the aforementioned memory 102 in order to access both at least one fluence map corresponding to the patient 104 and a deep learning model. Those skilled in the art will understand that fluence represents radiative flux integrated over time and comprises a fundamental metric in dosimetry (i.e., the measurement and calculation of an absorbed dose of ionizing radiation in matter and tissue). This fluence map, in turn, comprises a map of fluence values for various portions of the patient's body.

The aforementioned deep learning model is trained to deduce a leaf sequence for a multi-leaf collimator from a fluence map. Those skilled in the art understand that machine learning comprises a branch of artificial intelligence. Machine learning typically employs learning algorithms such as Bayesian networks, decision trees, nearest-neighbor approaches, and so forth, and the process may operate in a supervised or unsupervised manner as desired. Deep learning (also sometimes referred to as hierarchical learning, deep neural learning, or deep structured learning) is a subset of machine learning that employs networks capable of learning (typically unsupervised) from data that is unstructured or unlabeled. Deep learning architectures include deep neural networks, deep belief networks, recurrent neural networks, and convolutional neural networks. Many machine learning algorithms build a so-called “model” based on sample data, known as training data or a training corpus, in order to make predictions or decisions without being explicitly programmed to do so.

For the sake of an illustrative example, it is presumed in this description that the aforementioned deep learning model comprises a neural network model that was trained, at least in part, via a reinforcement learning method. It will also be presumed, and again for the sake of an illustrative example, that this neural network model comprises a convolutional neural network model and that the neural network was trained, at least in part, via supervised learning.

By one approach, this neural network model can be trained using a training corpus that includes fluence maps for each of a plurality of corresponding field/control points as pertain to a multi-leaf collimator and/or other features of a given radiation treatment platform.

As noted above, the neural network model was trained, at least in part, via a reinforcement learning method. In this illustrative example, the reinforcement learning method comprises a deep learning method. Those skilled in the art know that reinforcement learning comprises an area of machine learning using intelligent agents and determining how those agents should take actions in a particular environment in order to maximize some reward. Accordingly, this illustrative example provides for rewarding at least one agent during training. The latter may comprise calculating a reward based, at least in part, on how well a given created leaf sequence reproduces a target fluence.

At block 202, the control circuit 101 iteratively optimizes a radiation treatment plan to administer the therapeutic radiation 112 to the patient 104 by, at least in part, generating a leaf sequence for a multi-leaf collimator that comprises the aforementioned beam shaping apparatus 117 as a function of the aforementioned deep learning model and the aforementioned fluence map that corresponds to the patient 104. In particular, the control circuit 101 employs a plurality of agents to each separately use the deep learning model to each generate a leaf sequence for only a single leaf pair of the multi-leaf collimator.

Multi-leaf collimators are comprised of a plurality of individual parts (known as “leaves”) that are formed of a high atomic numbered material (such as tungsten) that can move independently in and out of the path of the radiation-therapy beam in order to selectively block (and hence shape) the beam. Typically the leaves of a multi-leaf collimator are organized in pairs that are aligned collinearly with respect to one another and which can selectively move towards and away from one another. A typical multi-leaf collimator has many such pairs of leaves, often upwards of twenty, fifty, or even one hundred such pairs.

By one approach, each of the aforementioned plurality of agents are identical to one another. By another approach (for example, when the multi-leaf collimator includes at least a first kind of leaf and a second kind of leaf, where the first and second kinds of leaves are different from one another (with respect, for example, to width, thickness, material composition, and so forth)), the plurality of agents can include some that are different from one another. For example, a first agent may generate leaf sequences for leaf pairs comprised of a first kind of leaf and a second agent may generate leaf sequences for leaf pairs comprised of a second, different kind of leaf.

So configured, these teachings provide for using reinforcement learning with a machine learning model by using reinforcement learning agents to observe and experiment with leaf sequencing and to assess relative success as a function of a reward mechanism that reflects how well a given leaf sequence succeeds with respect to achieving a particular fluence result.

For the sake of illustration, some more detailed examples will now be presented. It shall be understood that the details of these examples are intended to only serve in an illustrative role and are not intended to suggest any particular limitations as regards these teachings.

In this example, the basic reinforcement learning infrastructure divides the task into the agent and the environment. The agent interacts iteratively with the environment by deducing a proper action based on observation. During the training, the agent also gets a reward at each iteration. By maximizing the cumulative reward, the reinforcement agent policy for deducing the action from observation is modified. Once the agent training is adequate, the policy no longer changes, and the agent no longer requires the reward. Presuming a use of deep-reinforcement learning, by one approach the agent deduces the action by training a convolutional neural network.

This reinforcement learning-based leaf sequencing, in this example, employs a training environment where the plan creation optimization algorithm can be performed automatically for a representative set of cases (for example, various head and neck patient data). Once the agent (or agents) is trained and validated, it can be used to guide the optimization process for new patient cases. (While the agent may use deep Q reinforcement learning, other reinforcement learning methods (such as policy-gradient and actor-critic) may be employed as well as desired.)

By one approach the leaf sequencing agent converts a sector fluence to a corresponding leaf sequence, where the number of control points differ from 16 to 2 depending on the current multiresolution level. If desired, every multiresolution level can have a separate trained agent, and it is also possible that only certain multiresolution levels have agent-based leaf sequencing.

By one approach, a separate agent can be trained to deduce the monitor unit (MU) count of a single control point. These teachings would also accommodate utilizing a current MU count optimization algorithm.

These teachings will accommodate handling each leaf motion separately, so that a same agent is making multiple observations corresponding to each leaf-pair, or there could be a coordinated action based on a larger observation.

Since a given leaf-pair typically affects neighboring fluence rows (through a tongue-and-groove effect, these teachings will accommodate having each single leaf agent interact with some or all neighboring leaves. In such a case, a collaborative multi-agent approach can be implemented, such that agents controlling the neighboring leaves become part of the environment of the active agent.

By one approach, the reinforcement learning observation can be only comprise the target fluence row associated with a current leaf-pair. In a stacked (i.e., multi-layer) multi-leaf collimator design, each leaf-pair contributes to two fluence rows. These teachings will also accommodate adding fluence maps from a previous or next sequence, or leaf sequences from neighboring control points.

It is also possible to increase the data in the observation by providing full fluence rows, and/or by adding the current MU weights of different control points to the observation.

By one approach, the observation is the target fluence row as well as the importance of each fluence pixel as calculated by the optimizer using the second derivate of the cost function.

A simple reward calculation can be based on how well the created leaf sequence is actually reproducing the target fluence. If desired, however, these teachings will accommodate optimizing a cumulative reward rather than with respect to each iterative reward. So configured, the reinforcement learning agent learns also to benefit from future rewards, and part of the reward can be the final plan quality (for example, the value of the utility of the optimization).

By one approach, the reward can also receive a (weighted or unweighted) contribution from how well the leaf sequencing is satisfying the constraints and speed limits of the multi-leaf collimator.

In lieu of the foregoing, or in combination therewith, the reward can also have a component where the agent is penalized with respect to elongated optimization time. The penalization can be increased during the course of the optimization since it is less severe to violate the constraint while the optimization process is still on-going, and the solution is still likely to change. One approach to calculate the size of the maximum assigned penalty is to evaluate how large a change in fluence space is created when necessary changes to the leaf positions are done.

By one approach, one can define the reward at least partially as the weighted mean-square-difference between target fluence and the fluence generated by the proposed leaf sequence, and at least partially by penalizing leaf sequences that do not satisfy machine parameters and/or limits.

If desired, and as illustrated at optional block 203, these teachings will accommodate using the radiation treatment platform 114 that includes the aforementioned multi-leaf collimator to provide therapeutic radiation 112 to the patient 104 as a function of the optimized radiation treatment plan 113.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

What is claimed is:

1. An apparatus to facilitate administering therapeutic radiation to a patient, the apparatus comprising:
a memory having stored therein:
a fluence map corresponding to the patient;
a deep learning model trained to deduce a leaf sequence for a multi-leaf collimator from a fluence map, wherein the deep learning model comprises a neural network model that was trained, at least in part, via a reinforcement learning method;
a control circuit operably coupled to the memory and configured to iteratively optimize a radiation treatment plan to administer the therapeutic radiation to the patient by, at least in part, generating a leaf sequence as a function of the deep learning model and the fluence map that corresponds to the patient by employing a plurality of agents to each separately use the deep learning model to each generate a leaf sequence for only a single leaf pair of the multi-leaf collimator.

2. The apparatus of claim 1 wherein the neural network model was trained, at least in part, via a supervised learning method.

3. The apparatus of claim 1 wherein the neural network model was trained using a training corpus that includes fluence maps for each of a plurality of corresponding field/control points.

4. The apparatus of claim 1 wherein the neural network model comprises a convolutional neural network model.

5. The apparatus of claim 1 wherein the reinforcement learning method comprises a deep learning method.

6. The apparatus of claim 1 wherein the plurality of agents are each identical to one another.

7. The apparatus of claim 1 wherein the multi-leaf collimator is comprised of a first kind of leaf and a second kind of leaf, wherein the first and second kind of leaves are different from one another, and wherein the plurality of agents include a first agent that generates leaf sequences for leaf pairs comprised of the first kind of leaf and a second agent that generates leaf sequences for leaf pairs comprised of the second kind of leaf, wherein the first and second agents are different from one another.

8. The apparatus of claim 1 wherein the reinforcement learning method provides for rewarding an agent during training.

9. The apparatus of claim 1 wherein the reinforcement learning method provides for calculating a reward based, at least in part, on how well a created leaf sequence reproduces a target fluence.

10. The apparatus of claim 1 further comprising:
a radiation treatment platform that includes the multi-leaf collimator and that is configured to provide the therapeutic radiation to the patient as a function of the radiation treatment plan.

11. A method to facilitate administering therapeutic radiation to a patient, the method comprising:
accessing a memory having stored therein:
a fluence map corresponding to the patient;
a deep learning model trained to deduce a leaf sequence for a multi-leaf collimator from a fluence map, wherein the deep learning model comprises a neural network model that was trained, at least in part, via a reinforcement learning method;
by a control circuit operably coupled to the memory:
iteratively optimizing a radiation treatment plan to administer the therapeutic radiation to the patient by, at least in part, generating a leaf sequence as a function of the deep learning model and the fluence map that corresponds to the patient by employing a plurality of agents to each separately use the deep learning model to each generate a leaf sequence for only a single leaf pair of the multi-leaf collimator.

12. The method of claim 11 wherein the neural network model was trained, at least in part, via a supervised learning method.

13. The method of claim 11 wherein the neural network model was trained using a training corpus that includes fluence maps for each of a plurality of corresponding field/control points.

14. The method of claim 11 wherein the neural network model comprises a convolutional neural network model.

15. The method of claim 11 wherein the reinforcement learning method comprises a deep learning method.

16. The method of claim 11 wherein the plurality of agents are each identical to one another.

17. The method of claim 11 wherein the multi-leaf collimator is comprised of a first kind of leaf and a second kind of leaf, wherein the first and second kind of leaves are different from one another, and wherein the plurality of agents include a first agent that generates leaf sequences for leaf pairs comprised of the first kind of leaf and a second agent that generates leaf sequences for leaf pairs comprised of the second kind of leaf, wherein the first and second agents are different from one another.

18. The method of claim 11 wherein the reinforcement learning method provides for rewarding an agent during training.

19. The method of claim 11 wherein the reinforcement learning method provides for calculating a reward based, at least in part, on how well a created leaf sequence reproduces a target fluence.

20. The method of claim 11 further comprising:
by a radiation treatment platform that includes the multi-leaf collimator:
providing the therapeutic radiation to the patient as a function of the radiation treatment plan.
